(19) United States
(12) Patent Application Publication
(10) Pub. No.: US 2010/0297715 A1
(43) Pub. Date: Nov. 25, 2010

(54) METHOD FOR PRODUCING SUCCINIC ACID

(75) Inventors: Frederic Dehay, Laventie (FR); Laurent Segueilha, Saint Andre Lez Lille (FR); Olivier Calande, Armentieres (FR); Caroline Varlamoff, Annoeulin (FR)

Correspondence Address: YOUNG & THOMPSON, 209 Madison Street, Suite 500, Alexandria, VA, 22314, US

(73) Assignee: ROQUETTE FRERES, Lestrem (FR)

(21) Appl. No.: 12/747,987

(22) PCT Filed: Dec. 15, 2008
(86) PCT No.: PCT/FR2008/052300

§ 371 Date: Jun. 14, 2010

(30) Foreign Application Priority Data
Dec. 13, 2007 (FR) 07 59827
Feb. 18, 2008 (FR) 0851028

(51) Int. Cl. C12P 7/46; C07C 51/42

(52) U.S. Cl. 435/145; 562/593

(57) ABSTRACT

The invention relates to methods for producing succinic acid and/or succinate ions by fermentation under anaerobic conditions.

The present invention relates to methods for producing succinic acid and/or succinate ions by fermentation under anaerobic conditions.

Succinic acid (or butanedioic acid) is an organic acid with two carboxyl groups, of semi-structural formula COOH—CH₂—CH₂—COOH, which is involved in cell metabolism, as a metabolic intermediate of the Krebs cycle in the mitochondrion.

It has many applications in the cosmetics, food-processing, pharmaceutical and textile fields and in plastics. Thus, it is, for example, used as a synthesis intermediate for plastics, in the production of 1,4-butanediol, tetrahydrofuran and gamma-butyrolactone.

New products derived from succinic acid are constantly in development, including the development of polyesters.

Generally, succinic acid esters have the potential to be new “green” solvents which can replace solvents that are more harmful to humans and the environment.

The production of carboxylic acids, such as malic acid, succinic acid or fumaric acid, from renewable starting materials (in the case in point, via fermentation processes) is known to those skilled in the art.

Succinate is a metabolic intermediate in anaerobic fermentation by bacteria producing propionate, but these fermentation processes result in the production of very low yields and titers of succinic acid.

In recent years, many succinic acid-producing microorganisms have been isolated, for instance the anaerobic rumen bacteria Bacteroides ruminicola and Bacteroides amylophilus. However, organisms from the rumen are highly unstable in fermentation processes, and cannot therefore be used industrially for the production of succinic acid.

It has been known for a long time that a mixture of several acids, including succinic acid, is produced from E. coli fermentation in the presence of glucose and CO₂ as carbon substrates, as described by J L Stokes in 1949 “Fermentation of glucose by suspensions of Escherichia coli”, J. Bacteriol., 57: 147-158. However, for each mole of glucose fermented, only 0.3 to 0.4 mol of succinic acid is produced.

Studies have therefore been carried out on bacteria, in particular Escherichia coli that have been genetically modified so as to inactivate the metabolic pathways which consume the NADH needed for the production of succinic acid, and so as to activate the metabolic pathways for producing succinate (salt of succinic acid).

Specifically, the fermentative metabolic avenue which allows conversion of oxaloacetate to malate, then fumarate and, finally, succinate requires two mol of NADH per mole of succinate produced. The major metabolic bottleneck in the production of succinate is therefore the cellular bioavailability of NADH.

As a solution to this difficulty, document U.S. Pat. No. 7,223,567 describes the use of a recombinant Escherichia coli strain which overproduces succinate for the same available amount of NADH.

This Escherichia coli strain SBS550 MG pHL 413 exhibits inactivation of the products of the adhE and ldhA genes (involved in the pathways which consume NADH) and inactivation of the products of the ack-pta genes and of the iclR gene (activating the glyoxylate pathway), and contains a plasmid vector which overexpresses an exogenous PYC gene.

The article by Sanchez et al. (titled “Novel pathway engineering design of the anaerobic central metabolic pathway in Escherichia coli to increase succinate yield and productivity” in Metabolic Engineering 7 (2005) 229-239), U.S. Pat. No. 7,223,567 and U.S. patent application US 2005/0042736 have developed new culturing and production conditions associated with this strain, to improve its succinic aid production yields.

Those skilled in the art are constantly searching for new improved methods for producing succinic acid. In particular, those skilled in the art seek to optimize the yield and the productivity obtained. Moreover, conventional fermentation methods result in considerable amounts of carbon dioxide waste being released into the atmosphere, which is quite obviously undesirable.

According to one aspect, the present invention relates to a method for producing succinic acid and/or succinate ions by anaerobic fermentation of an Escherichia coli strain, comprising:
(A) a step of fermentation of a carbon source in a fermenter, with CO₂ being supplied, carried out with fermenter vents closed such that the supply of CO₂ is controlled by the consumption of CO₂ by the strain; followed, before complete depletion of the carbon source, by
(B) a step of fermentation of the remaining carbon source, without CO₂ being supplied, so as to consume the residual CO₂.

Those skilled in the art are familiar with fermentation techniques (as in particular described in Fermentation & Biochemical Engineering Handbook: principles, process design & equipment, 2nd ed 1996 by Henry C. Vogel and Celeste L. Todaro).

Fermentation is a biochemical reaction which generally consists in releasing energy or in producing certain metabolites of interest, from an organic substrate under the action of microbial enzymes.

Fermentation is generally carried out in devices (fermenters) suitable for the fermentation process, i.e. suitable for culturing microorganisms under the desired conditions (devices making it possible, where appropriate, to control the gas equilibria of the culture medium, in particular by means of gas inlet and/or outlet pipes, vents, etc; devices making it possible to introduce culture medium and other substances; devices making it possible to control, regulate, modify other types of parameters, such as stirring, temperature, pH, etc).

Those skilled in the art are also familiar with fermentation under anaerobic conditions. According to the present invention, this denotes culture conditions in the absence of oxygen. Preferably, anaerobic culture conditions are culture conditions in the presence of carbon dioxide. According to one embodiment, the anaerobic fermentation conditions in the presence of CO₂ and/or with CO₂ being supplied are CO₂-saturation fermentation conditions.

The expression “before complete depletion of the carbon source” during step (A) is intended to mean a moment in step (A) where the fermentation medium contains a residual amount of carbon source that can be entirely converted, by the strain, to succinic acid and/or succinate by virtue of the CO₂ available in solution at this moment (in the form of dissolved CO₂ or of HCO₃⁻).

During step (A), since the fermenter vent(s) is (are) closed and the CO₂ feed to the fermenter is maintained, the supplying of CO₂ is carried out batchwise, automatically adjusted according to the consumption of CO₂ by the strain for producing succinic acid and/or succinate.

The term “automatically adjusted” is intended to mean, given the thermodynamic equilibrium between the liquid phase (fermentation medium) and the gas phase (“atmosphere”) present in the fermenter (vent(s) closed), that the supply of a given amount of CO₂ can occur only subsequent to the consumption of an equivalent amount by the strain through fermentation (and therefore the concomitant production of succinic acid and/or succinate).

During step (B), there is no supply of CO₂, which means that the supplying of CO₂ carried out during step (A) is interrupted. This can in particular be carried out by cutting off the CO₂ feed.

Advantageously, according to the invention, the fermentation during step (B) consumes the CO₂ (dissolved residual) and the HCO₃⁻ ions present in the fermentation medium.

According to one preferred embodiment, before the start of step (A), the supplying of CO₂ is carried out by injection, with fermenter vents open, so as to reach saturation of the fermentation medium with CO₂.

By way of example, the CO₂ can be introduced by injection at a flow rate of 0.15-0.40 vvm (volume of CO₂ per volume of culture per minute), preferably 0.3 vvm.

The expression “fermentation medium saturated with CO₂” is intended to mean that the culture medium contains the maximum amount of CO₂ that can be dissolved therein under the corresponding conditions (temperature, pH, etc). For example, this can correspond to a concentration of 1-2 g/l, for example of the order of 1.5 g/l at 37° C., pH 7.

According to one embodiment, step (A) and/or step (B) is (are) carried out at a pH in a range of 6.0-7.0, preferably 6.4-6.8, preferably 6.5-6.6.

According to one embodiment, the carbon source is glucose.

According to one embodiment, at the start of step (A), the fermentation medium comprises 15-40 g/l, preferably 15-25 g/l, preferably 15-20 g/l of glucose.

According to one embodiment, at the start of step (B), the fermentation medium comprises 2-6 g/l, preferably approximately 4 g/l of glucose.

According to one preferred embodiment, the Escherichia coli strain is a strain which has the genotype ΔadhE ΔldhA ΔiclR Δackpta PYC. This genotype advantageously makes it possible to promote the production of succinic acid by fermentation in the presence of CO₂. The symbol Δ indicates that the gene in question has been inactivated, for example by mutation, deletion, interruption, insertion or down-regulation, for example by introducing a stop codon, insertion or deletion resulting in a change of reading frame, a point mutation, etc.

The ΔadhE ΔldhA ΔiclR Δackpta PYC genotype therefore corresponds to:
ΔadhE: inactivation of alcohol dehydrogenase;
ΔldhA: inactivation of lactate dehydrogenase;
ΔiclR: inactivation of isocitrate lyase (also known as aceA);
Δackpta: inactivation of acetate kinase-phosphotransacetylase;
PYC: expression of a pyruvate carboxylase gene. This indicates that the strain expresses the PYC gene, for example by virtue of a transformation with a plasmid carrying a functional copy of this gene, or by genomic integration of a functional copy of PYC. The PYC gene is advantageously the Lactococcus lactis pyc gene.

According to one very preferred embodiment, the Escherichia coli strain is the SBS550MG-pHL413 strain. This strain is described in Sanchez et al., Metabolic Engineering, 7 (2005) 229-239, and in documents U.S. Pat. No. 7,223,567 and US 2005/0042736.

According to one aspect, the present invention relates to a method for producing succinic acid, comprising:
(a) a step of culturing an Escherichia coli strain under aerobic conditions, during which the pH is regulated by addition, to the culture medium, of a magnesium compound,
(b) a step of producing succinate ions by fermentation of the strain cultured in step (a), under anaerobic conditions in the presence of CO₂,
(c) a step of converting the succinate ions formed in step (b) into succinic acid.

Those skilled in the art are also familiar with fermentation and culturing under aerobic conditions. According to the present invention, this denotes culture conditions in the presence of oxygen. According to one embodiment, the oxygen comes from the atmosphere.

In step (a), there is thus growth and propagation of the E. coli strain. There is thus production of biomass, i.e. an increase in the cell population. This step can typically comprise preculture substeps.

According to the present invention, the term “regulating pH” is intended to mean the action of maintaining the pH value of the culture medium within a certain range or selection of values. According to the invention, the pH can be regulated in various ways:
regulation within a range: the pH value is maintained within a certain range of values. The pH value can then vary over time, without however departing from the range under consideration;
“low-point” regulation: the pH value is maintained above a threshold value. The pH value can then vary over time, without however dropping below the threshold value;
regulation at a single value: the pH value is maintained at this value constantly over time.

The term “addition of a compound” is intended to mean the introduction of the compound into the culture medium. The addition can be carried out according to various methods: addition of a suspension and/or addition of a solution and/or addition of a solid (for example, in powder form).

According to one embodiment, the magnesium compound of step (a) is chosen from magnesium oxide, magnesium hydroxide and magnesium carbonate.

The magnesium oxide, the magnesium hydroxide or the magnesium carbonate can be added in powder form or in the form of suspensions, typically of an aqueous suspension, for example at concentrations of 20% to 30% w/v.

According to one embodiment, steps (a) and/or (b) is (are) carried out in a medium containing the carbon source used, typically glucose, and in particular at concentrations of 10-30 g/l, for example 20 g/l.

According to one embodiment, during step (b), the pH is regulated by the addition, to the fermentation medium, of a compound chosen from the group constituted of magnesium compounds (for example, chosen from magnesium oxide, magnesium hydroxide and magnesium carbonate), calcium compounds (for example, chosen from calcium oxide, calcium hydroxide and calcium carbonate), potassium compounds (for example, chosen from potassium hydroxide and potassium carbonate), ammonium compounds (for example, chosen from ammonium hydroxide and ammonium carbonate) and sodium compounds (for example, chosen from sodium hydroxide and sodium carbonate), and mixtures thereof.

According to one embodiment, step (b) is carried out at a pH within a range of 6.0-7.0, preferably 6.4-6.8.

According to one embodiment, step (c) comprises an acidification. The acidification can in particular be carried out by the addition of at least one acid chosen from ortho-phosphoric acid, oxalic acid and sulfuric acid.

According to one embodiment, during step (b), the pH is regulated by the addition, to the fermentation medium, of a compound chosen from the group constituted of magnesium compounds, thus forming magnesium succinate.

In this case, step (c) can comprise:
(c-1) a step of converting magnesium succinate formed in step (b) into sodium succinate, and
(c-2) a step of converting, by bipolar electrodialysis, the sodium succinate formed in step (c-1) into succinic acid.

Step (c-1) can typically be carried out by adding sodium carbonate. The carbonate can be added in the form of a solution or of a powder, typically of an aqueous solution, for example at concentrations of 1 to 2M. The magnesium carbonate, which is insoluble, precipitates. The magnesium carbonate can be treated in an oven at high temperature, for example an oven at >700° C. This results in MgO and CO₂, at least one of which can be recycled.

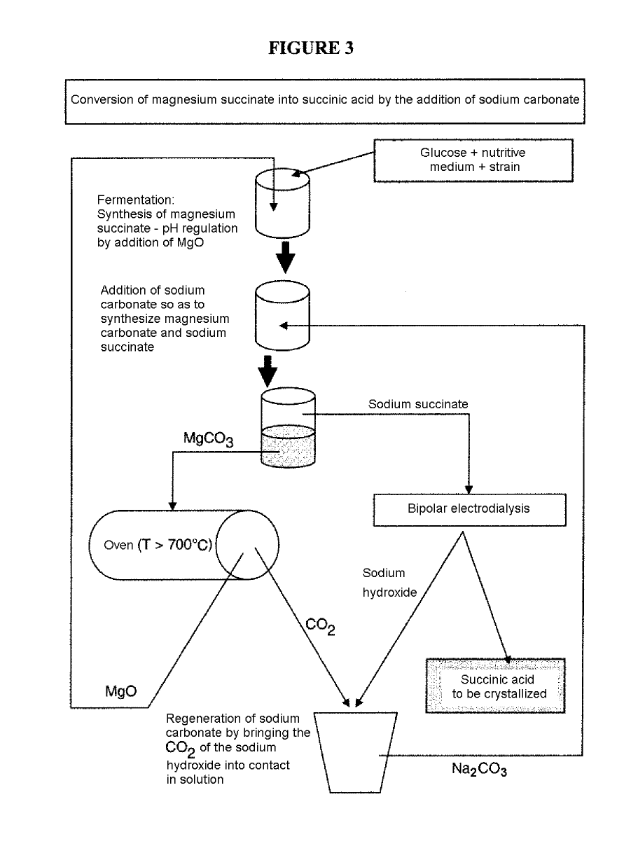

The magnesium carbonate can alternatively be recovered as such. The sodium succinate can advantageously be treated by bipolar electrodialysis (which is not the case with magnesium succinate), giving sodium hydroxide and succinic acid, which can be crystallized. The bipolar electrodialysis technique is, moreover, well known to those skilled in the art. The sodium hydroxide produced can, where appropriate, be reconverted, with the CO₂ previously emitted from the high-temperature oven, so as to form sodium carbonate. All the steps are represented in FIG. 3.

According to one preferred embodiment, the Escherichia coli strain is a strain which has the ΔadhE ΔldhA ΔiclR Δackpta PYC genotype. According to one very preferred embodiment, the Escherichia coli strain is the SBS550MG-pHL413 strain.

According to another aspect, the present invention relates to a method for producing succinic acid comprising:
(i) a step of producing magnesium succinate by fermentation, under anaerobic conditions, of an Escherichia coli strain in the presence of CO₂, during which the pH is regulated by the addition, to the fermentation medium, of a magnesium compound, and
(ii) a step of converting the magnesium succinate formed in step (i) into succinic acid.

The expressions “regulating the pH” and “addition of a compound” are defined above.

According to one embodiment, the magnesium compound of step (i) is chosen from magnesium oxide, magnesium hydroxide and magnesium carbonate. Preferably, it is magnesium oxide (magnesia, of formula MgO).

The magnesium oxide, the magnesium hydroxide or the magnesium carbonate can be added in powder form or in the form of a suspension, typically of an aqueous suspension, for example at concentrations of 20% to 30% w/v.

According to one embodiment, step (i) is carried out at a pH within the range of 6.0-7.0, preferably 6.4-6.8, preferably 6.5-6.6.

According to one embodiment, steps (i) and (ii) are carried out in a medium containing the carbon source used, typically glucose, and in particular at concentrations of 10-30 g/l, for example 20 g/l.

According to one embodiment, step (ii) comprises an acidification. This acidification can be carried out in various ways. According to one embodiment, the acidification is carried out by the addition of at least one acid chosen from ortho-phosphoric acid, oxalic acid and sulfuric acid. These acids can be added in pure form or in the form of concentrated aqueous solutions.

According to another embodiment, step (ii) comprises: (ii-a) a step of converting the magnesium succinate formed in step (i) into sodium succinate, and (ii-b) a step of converting, by bipolar electrodialysis, the sodium succinate formed in step (ii-a) into succinic acid.

These steps were described above for steps (c-1) and (c-2).

According to one preferred embodiment, the Escherichia coli strain is a strain which has the ΔadhE ΔldhA ΔiclR Δackpta PYC genotype. According to one very preferred embodiment, the Escherichia coli strain is the SBS550MG-pHL413 strain.

According to another aspect, the present invention relates to a method for obtaining succinic acid, comprising:
a method for producing succinic acid and/or succinate ions, for example chosen from those described above;
a step of acidifying the succinate ions so as to give succinic acid, for example by addition of a strong acid to the must,
optionally, a step of purifying the succinic acid; and
a step of crystallizing the succinic acid.

According to one embodiment, in all the methods described above, the purification step comprises an ethanolic purification which is carried out as follows:
filtration (removal of a protein precipitate) of the acidified must, for example through a Büchner funnel and/or through filtering earth,
optionally, concentration of the filtrate by evaporation under vacuum (preferably, according to a concentration factor between approximately 2 and 8),
addition of ethanol, for example of 95% ethanol, in a 1/1 to 5/1 ratio, so as to cause precipitation of the salts (the succinic acid remains soluble),
separation of the saline precipitate by filtration, for example through a membrane,
recovery of the ethanol by evaporation under vacuum,
treatment on active carbon, and then plate filtration and filtration through filtering earth.

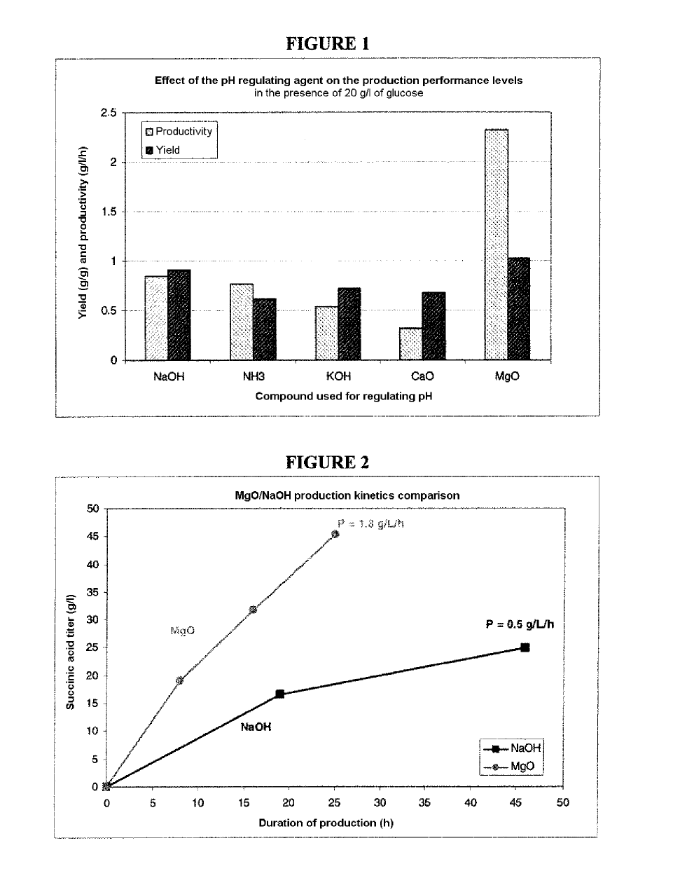

FIG. 1 illustrates the performance levels for production of succinic acid by anaerobic fermentation as a function of the compound used to regulate pH.

FIG. 2 compares the production kinetics according to whether MgO or NaOH is used to regulate pH.

FIG. 3 represents an embodiment for producing succinic acid: conversion of magnesium succinate into succinic acid by the addition of sodium carbonate.

The invention is illustrated by the exemplary embodiments below, which are nonlimiting.

### EXAMPLES

### Example 1

### Production of Succinic Acid by Anaerobic Fermentation According to Two Different Methods of Supplying CO₂ with Fermenter Vent Opened or Closed

The method for producing succinic acid comprises:
a phase of preculturing in Erlenmeyer flask,
a phase of culturing under aerobic conditions in a culture medium comprising corn steep as nitrogen source and glucose as carbon source, this phase allowing the production of biomass, and
an anaerobic phase allowing the production per se of succinic acid.

The phases under aerobic and anaerobic conditions are carried out in the same fermenter. The strain used is the SBS550MG-pHL413 strain.

### Aerobic Phase:

The SBS550MG-pHL413 strain is precultured in an Erlenmeyer flask for 17 h at 37° C., with shaking at 125 rpm. 400 ml of medium are inoculated with the strain in a 2-liter Erlenmeyer flask with 2 baffles.

The composition of this preculture medium is the following:

| | |
|---|---|
| Tryptone | 10 g/l |
| Yeast extract | 5 g/l |
| NaCl | 10 g/l |
| Antibiotics (ampicillin, | 67 mg/l |
| carbenicillin, oxacillin) | |

The strain thus precultured is placed in a 15 l fermenter in a culture medium of which the composition is the following:

| Salts and antibiotics: | g/l |
|---|---|
| (NH₄)₂SO₄ | 0.25 |
| K₂HPO₄ | 0.7 |
| KH₂PO₄ | 1.2 |
| KCl | 2 |
| CaCl₂ | 0.2 |
| MgSO₄ | 0.25 |
| Ampicillin | 0.067 |
| Biotin | 0.001 |
| Thiamine | 0.001 |

Glucose: 2 g/l at the start + 2 g/l when the first 2 g/l are consumed
Corn steep: 60 g/l

The inoculum obtained by preculturing in an Erlenmeyer flask represents 3% of the total volume of the medium cultured in the fermenter.

The culture conditions during the aerobic phase are a temperature of 37° C., stirring at 500 rpm, an aeration of 1 vvm and no pH regulation (the pH is simply adjusted to 7.5 before sterilization of the medium).

### Anaerobic Phase:

Protocol with Continuous Supply of CO₂
The following are added to the medium: glucose: 20 g/l at the start+15 g/l at 24 h+4 g/l at 50 h.
The fermentation is carried out at pH 6.4 with continuous injection of CO₂ at a flow rate of 0.3 vvm (l/l/min), at 37° C., fermenter vent open, with stirring at 250 rpm.
It comes to an end in 63.5 h with a final succinic acid titer of 30 g/l in the culture medium.
The overall amount of CO₂ consumed comes to (0.3 vvm×60 min×63.5 h/22.4 mol/l×44 g/mol) 2245 g/l, i.e. 73 g/g of succinic acid formed.
Moreover, a concentration of 2 g/l of HCO₃⁻ (corresponding to 1.5 g/l of CO₂) is present at the end of fermentation in the culture medium.

Protocol According to the Invention

The fermentation protocol is identical to that above, except that
at the beginning of the anaerobic phase, CO₂ is introduced into the fermenter at a flow rate of 0.3 vvm for 1 minute so as to drive off the residual air resulting from the aerobic phase,
the pressure of the CO₂ system is reduced to 0.4 bar,
the fermenter vent is hermetically closed so as to prevent the CO₂ from leaving,
thus, the injection of CO₂ is accurately adjusted to its consumption throughout the fermentation,
the injection of CO₂ is stopped when the residual concentration of glucose reaches 4 g/l, so as to consume the residual HCO₂⁻ dissolved in the medium.

Results According to the Invention
Final concentration of succinic acid produced in the culture medium at the end of fermentation: 30 g/l, which is identical to the concentration obtained with continuous supply of CO₂; concentration of residual HCO₂⁻ in the culture medium at the end of fermentation: 0.3 g/l (which represents an 85% reduction compared with the concentration obtained with continuous supply of CO₂);
consumption of CO₂: 0.59 g/l at the start+6 g/l bonded on the succinic acid, i.e. 0.2 g/g of acid (which represents a 99.7% reduction compared with the concentration obtained with continuous supply of CO₂).

Thus, advantageously according to the invention, while maintaining the succinic acid production yield, substantial amounts of carbon dioxide waste are avoided:
working in a closed reactor naturally limits the waste, and
moreover, a decrease in the concentration of residual HCO₃⁻ in the culture medium at the end of fermentation is observed. However, the acidification of the residual HCO₃⁻ in the culture medium at the end of fermentation results in CO₂ being given off.

### Example 2

### Production of Succinic Acid by Anaerobic Fermentation with an Inorganic Medium and Regulation of pH with Various Compounds, at Least One of which being Magnesium-Based

The strain used is the SBS550MG-pHL413 strain.

During the phase of fermentation under anaerobic conditions, the pH is regulated at a value of 6.75 using various compounds: NaOH, NH₃, KOH, CaO or MgO.

The protocol scheme is the following:
preculturing in Erlenmeyer flask;
subculturing in a fermenter;
production in a fermenter: 2 phases:
   aerobic phase: production of biomass,
   anaerobic phase: production of succinic acid in the presence of CO₂.

Each step is detailed below:

### Preculturing in an Erlenmeyer Flask

| Medium | g/l |
|---|---|
| Tryptone | 10 |
| Yeast extract | 5 |
| NaCl | 10 |
| KH₂PO₄ | 3 |
| Na₂HPO₄ | 6 |
| NH₄Cl | 1 |
| MgSO₄•7H₂O | 0.25 |
| NaCl | 0.5 |
| Antibiotics (ampicillin, | 67 mg/l |
| carbenicillin, oxacillin) | |

incubation at 37° C. for <24 h;
shaking: 125 rpm;
volume: 500 ml in a 2 l Erlenmeyer flask.

### Subculturing in a Fermenter

| Medium | g/l |
|---|---|
| Glucose | 10 |
| (NH₄)₂HPO₄ | 6 |
| K₂HPO₄ | 0.5 |
| K₂SO₄ | 1 |
| KCl | 2 |
| MgSO₄•7H₂O | 2 |
| Trace elements, vitamins | |
| and antibiotics | mg/l |
| FeSO₄•7H₂O | 60 |
| CaCl₂•2H₂O | 30 |
| ZnSO₄•7H₂O | 4 |
| CuCl₂•2H₂O | 2 |
| MnSO₄•H₂O | 20 |
| CoCl₂•6H₂O | 8 |
| H₃BO₃ | 1 |
| Na₂MoO₄•2H₂O | 0.4 |
| Biotin | 1 |
| Thiamine | 1 |
| Ampicillin | 67 |

inoculum 6%;
37° C., stirring: 450 rpm, aeration: 1 vvm;
pH regulated at 6.75 with 5N NaOH;
duration: >20 h.

### Production in a Fermenter: 2 Phases

Aerobic Phase: Production of Biomass

Medium

| Salts: | g/l |
|---|---|
| (NH₄)₂HPO₄ | 6 |
| K₂HPO₄ | 0.5 |
| K₂SO₄ | 1 |
| KCl | 2 |
| MgSO₄•7H₂O | 2 |

Glucose: 10 g/l at the start + 10 g/l when the first 10 g/l are consumed

Trace elements and vitamins: idem subculture;
inoculum 13%;
37° C., stirring: 450 rpm, aeration: 1 vvm;
regulation of pH at 6.75 by addition of 5N NaOH, respectively 28% w/v NH₃, respectively 5N KOH, respectively 20% w/v CaO, respectively 20% w/v MgO.
   Anaerobic Phase: Succinic Production with Supply of CO₂
Addition of glucose 20 g/l;
injection of CO₂ at 0.2 vvm;
37° C., stirring: 250 rpm;
regulation of pH at 6.75 by addition of 5N NaOH, respectively 28% w/v NH₃, respectively 5N KOH, respectively 20% w/v CaO, respectively 20% w/v MgO.

For each protocol, the amount of succinic acid produced is measured by HPLC.

The results are represented in FIG. 1, with the productivities and the yields obtained over short production phases corresponding to the consumption of 20 g/l of glucose.

Surprisingly and advantageously according to the invention, for the regulation of pH during the anaerobic fermentation, the use of MgO gives by far the best performance levels, with a yield greater than 100% and a productivity by volume that is 2.5 times greater than that obtained with the most effective of the other bases (NaOH).

The table below completes the comparison by showing that the use of MgO also gives the best biomass production yield, and the lowest synthesis of co-products.

| | | Productivity | | Specific | |
|---|---|---|---|---|---|
| | Biomass | by volume | Succinic acid | productivity | Co-products |
| | yield | succinic acid | yield | succinic acid | Malic acid + |
| | Y x/g | PV sa | Y sa/g | PS sa | pyruvic acid |
| | % glucose | g/l/h | % glucose | g/g/h | % |
| | aerobic | anaerobic | anaerobic | anaerobic | succinic |
| NaOH | 25 | 0.9 | 91 | 0.24 | 26 |
| NH₃ | 29 | 0.8 | 62 | 0.20 | 22 |
| KOH | 25 | 0.5 | 72 | 0.16 | 48 |
| CaO | 32 | 0.3 | 68 | 0.12 | 50 |
| MgO | 35 | 2.3 | 103 | 0.48 | 21 |

sa = succinic acid

### Production Kinetics: Comparison of the Regulation of pH by Addition of MgO and by Addition of NaOH

FIG. 2 compares the change in the succinic acid titer obtained by prolonging the production phases in the presence of sodium hydroxide or of magnesia.

This comparison reveals another unexpected advantage of the use of MgO: the low degree of slowing of the kinetics during the accumulation of succinic acid. Advantageously, the use of MgO makes it possible to increase the productivity and the rate of production of succinic acid compared with the use of NaOH. In addition, the use of MgO makes it possible to reach succinic acid concentrations (titers) of greater than 50 g/l.

### Example 3

### Production of Succinic Acid and Regulation of pH in the Aerobic Growth Phase Using a Magnesium Compound

The strain used is the SBS550MG-pHL413 strain.

The protocol used for the preculturing and the subculturing is identical to that described in example 2.

For the production in a fermenter, according to the invention, the regulation of pH during the aerobic culture phase (growth of the strain, production of biomass) is carried out using MgO, whereas the regulation of pH during the phase of succinate production under anaerobic conditions is carried out using sodium hydroxide.

The other working conditions are identical to those of example 2.

This is summarized by the notation “MgO then NaOH” which indicates that the aerobic culturing step is carried out with the addition of MgO, followed by an anaerobic fermentation step with the addition of NaOH.

The table below compares the results obtained under these pH regulation conditions (“MgO then NaOH”) with those obtained when only MgO or only NaOH is used in the two phases (“MgO then MgO” or “NaOH then NaOH”).

| | | Productivity | | Specific | |
|---|---|---|---|---|---|
| | Biomass | by volume | Succinic acid | productivity | Co-products |
| | yield | succinic acid | yield | succinic acid | Malic acid + |
| | Y x/g | PV sa | Y sa/g | PS sa | pyruvic acid |
| | % glucose | g/l/h | % glucose | g/g/h | % |
| | aerobic | anaerobic | anaerobic | anaerobic | succinic |
| “MgO then | 35 | 1.8 | 95 | 0.37 | 20 |
| NaOH” | | | | | |
| “MgO then | 35 | 2.3 | 103 | 0.48 | 21 |
| MgO” | | | | | |
| “NaOH then | 25 | 0.9 | 91 | 0.24 | 26 |
| NaOH” | | | | | |

sa = succinic acid

These results show that it is possible to obtain a positive effect on the performance levels in the production phase by regulating the pH using MgO only during the growth phase.

### Example 4

### Obtaining Succinic Acid by Anaerobic Fermentation and Acidification

The strain used is the SBS550MG-pHL413 strain. The method for producing succinic acid of example 2 (with MgO as pH regulating agent) is followed by a step of acidification by addition of various acids:
Ortho-phosphoric acid: Purification by formation and precipitation of magnesium phosphate tribasic (highly insoluble). 1 mol of H₃PO₄ is added per mole of succinic in the aqueous phase. This results in the formation of highly soluble magnesium phosphate monobasic.
Oxalic acid: Purification by formation and precipitation of highly insoluble magnesium oxalate. There is no loss of succinic acid. The succinic acid freed of its counterion is rapidly obtained.

### Example 5

### Obtaining Succinic Acid by Anaerobic Fermentation and Conversion into Sodium Succinate

The strain used is the SBS550MG-pHL413 strain. The method for producing succinic acid of example 2 (with MgO as pH regulating agent) is followed by a step of formation of magnesium carbonate and of sodium succinate, by addition of sodium carbonate.

The magnesium carbonate, which is insoluble, precipitates. There are no losses of succinic acid. The magnesium carbonate can be treated in an oven at high temperature, for example an oven at >700° C. This results in MgO and CO₂, at least one of which can be recycled. The magnesium carbonate can then be advantageously recovered.

The sodium succinate can be treated by bipolar electrolysis, giving sodium hydroxide and succinic acid, which can be crystallized. This sodium hydroxide can be reconverted, with the CO₂ previously emitted from the high-temperature oven, so as to form sodium carbonate.

All the steps are represented in FIG. 3.

### Example 6

### Obtaining Succinic Acid by Anaerobic Fermentation, Ethanolic Purification and Crystallization

The method for producing succinic acid in example 1 (with NaOH as pH regulating agent) is followed by a step of ethanolic purification as described below:
centrifugation of the fermentation medium (must) (5000 g, 15 min, 20° C.) (elimination of the biomass),
addition of 95% sulfuric acid to the supernatant until a pH of 1.5 is obtained,
filtration of the must through a Büchner funnel with a Seitz EK plate and FW20 filtering earth (elimination of a protein precipitate),
concentration of the filtrate by evaporation under vacuum (concentration factor fluctuates between approximately 2 and 8),
addition of 95% ethanol, 2 volumes of ethanol per volume of the concentrated filtrate so as to cause precipitation of the salts (the succinic acid remains soluble),
separation of the saline precipitate by filtration through a 3-micron millipore membrane,
recovery of the ethanol by evaporation under vacuum,
treatment with active carbon (2%, on a dry weight basis, of Pureflow C—80° C.—1 hour) then filtration through a Seitz EK plate and FW20 filtering earth,
evapo-crystallization (Tp water bath 75° C.—residual pressure 60 mbar—dry matter of the crystalline cooked mass of approximately 50%),
cooling of the crystalline cooked mass with stirring at 20° C.,
separation of the crystals by filtration through a 3-micron millipore membrane,
clarifying of the crystals with demineralized water,
drying of the crystals overnight at 60° C. under vacuum.

1. A method for producing succinic acid and/or succinate ions by fermentation, under anaerobic conditions, of an Escherichia coli strain, comprising:
(A) a step of fermentation of a carbon source in a fermenter, with CO₂ being supplied, carried out with fermenter vents closed such that the supply of CO₂ is controlled by the consumption of CO₂ by the strain; followed, before complete depletion of the carbon source, by
(B) a step of fermentation of the remaining carbon source, without CO₂ being supplied, so as to consume the residual CO₂.

2. The method as claimed in claim 1, in which, at the start of step (A), the fermentation medium is saturated with CO₂.

3. The method as claimed in claim 1, in which step (A) and/or step (B) is (are) carried out at a pH within a range of 6.0-7.0, preferably 6.4-6.8.

4. The method as claimed in claim 1, in which the carbon source is glucose, and in which:
at the start of step (A), the fermentation medium comprises 15-40 g/l of glucose, and
at the start of step (B), the fermentation medium comprises 2-6 g/l of glucose.

5. The method as claimed in claim 1, in which the Escherichia coli strain has the ΔadhE ΔldhA ΔiclR Δackpta PYC genotype; preferably, the Escherichia coli strain is the SBS550MG-pHL413 strain.

6. A method for obtaining succinic acid, comprising:
a method for producing succinic acid and/or succinate ions as claimed in claim 1;
where appropriate, acidification of the succinate ions so as to give succinic acid;
a step of purifying the succinic acid, preferably an ethanolic purification; and
optionally, a step of crystallizing the succinic acid.

7. A method for producing succinic acid, comprising:
(a) a step of culturing an Escherichia coli strain under aerobic conditions, during which the pH is regulated by the addition, to the culture medium, of a magnesium compound,
(b) a step of producing succinate ions by fermentation of the strain cultured in step (a), under anaerobic conditions in the presence of CO₂,
(c) a step of converting the succinate ions formed in step (b) into succinic acid.

8. The method as claimed in claim 7, in which, in step (a), the magnesium compound is chosen from magnesium oxide, magnesium hydroxide and magnesium carbonate.

9. The method as claimed in claim 7, in which, during step (b), the pH is regulated by the addition, to the fermentation medium, of a compound chosen from the group constituted of magnesium compounds, calcium compounds, potassium compounds, ammonium compounds and sodium compounds, and mixtures thereof.

10. The method as claimed in claim 7, in which step (b) is carried out at a pH within a range of 6.0-7.0, preferably 6.4-6.8.

11. The method as claimed in claim 7, in which step (c) comprises an acidification.

12. The method as claimed in claim 11, in which the acidification is carried out by the addition of at least one acid chosen from ortho-phosphoric acid, oxalic acid and sulfuric acid.

13. The method as claimed in claim 7, in which, during step (b), the pH is regulated by the addition, to the fermentation medium, of a compound chosen from the group constituted of magnesium compounds, thus forming magnesium succinate, and step (c) comprises:
(c-1) a step of converting magnesium succinate formed in step (b) into sodium succinate, and
(c-2) a step of converting, by bipolar electrodialysis, the sodium succinate formed in step (c-1) into succinic acid.

14. The method as claimed in claim 7, in which the Escherichia coli strain has the ΔadhE ΔldhA ΔiclR Δackpta PYC genotype; preferably, the Escherichia coli strain is the SBS550MG-pHL413 strain.

15. A method for obtaining succinic acid, comprising:
a method for producing succinic acid as claimed in claim 7;
a step of purifying the succinic acid, preferably an ethanolic purification; and
optionally, a step of crystallizing the succinic acid.

16. A method for producing succinic acid, comprising:
(i) a step of producing magnesium succinate by fermentation, under anaerobic conditions, of an Escherichia coli strain in the presence of CO₂, during which the pH is regulated by the addition, to the fermentation medium, of a magnesium compound, and
(ii) a step of converting the magnesium succinate formed in step (i) into succinic acid.

17. The method as claimed in claim 16, in which, in step (i), the magnesium compound is chosen from magnesium oxide, magnesium hydroxide and magnesium carbonate.

18. The method as claimed in claim 16, in which step (i) is carried out at a pH within a range of 6.0-7.0, preferably 6.4-6.8.

19. The method as claimed in claim 16, in which step (ii) comprises an acidification.

20. The method as claimed in claim 19, in which the acidification is carried out by the addition of at least one acid chosen from ortho-phosphoric acid, oxalic acid and sulfuric acid.

21. The method as claimed in claim 16, in which step (ii) comprises:
(ii-a) a step of converting the magnesium succinate formed in step (i) into sodium succinate, and
(ii-b) a step of converting, by bipolar electrodialysis, the sodium succinate formed in step (ii-a) into succinic acid.

22. The method as claimed in claim 16, in which the Escherichia coli strain has the ΔadhE ΔidhA ΔiclR Δackpta PYC genotype; preferably, the Escherichia coli strain is the SBS550MG-pHL413 strain.

23. A method for obtaining succinic acid, comprising:
a method for producing succinic acid as claimed in claim 16;
a step of purifying the succinic acid, preferably an ethanolic purification; and
optionally, a step of crystallizing the succinic acid.

24. A method for purifying succinic acid and/or succinate ions from a fermentation must, comprising:
a step of acidifying the succinate ions so as to give succinic acid, by the addition of sulfuric acid to the must,
a step of purifying the succinic acid by the addition of ethanol; and
optionally, a crystallization.
